(19) United States
(12) Patent Application Publication
(10) Pub. No.: US 2026/0263418 A1
(43) Pub. Date: Sep. 10, 2026

(54) AGENTS FOR ENHANCING DOPAMINE RELEASE, IMPROVING MOTOR CONTROL FUNCTION, IMPROVING MOTOR LEARNING FUNCTION AND IMPROVING ACTIVITY MOTIVATION

(71) Applicant: MEIJI CO., LTD., Tokyo (JP)

(72) Inventors: Koji NONAKA, Tokyo (JP); Kyosuke NAKAYAMA, Tokyo (JP); Chiaki SANBONGI, Tokyo (JP)

(73) Assignee: MEIJI CO., LTD., Tokyo (JP)

(21) Appl. No.: 19/469,695

(22) PCT Filed: Mar. 26, 2024
(86) PCT No.: PCT/JP2024/012048

§ 371 Date: Sep. 26, 2025

(30) Foreign Application Priority Data
Mar. 29, 2023 (JP) 2023-054205

(51) Int. Cl. A61K 31/405

(52) U.S. Cl. CPC A61K 31/405

(57) ABSTRACT

An object of the present invention is to provide a novel agent for enhancing dopamine release, a novel agent for improving motor control function, a novel agent for improving motor learning function and a novel agent for improving activity motivation. To achieve the object, the present invention provides an agent for enhancing dopamine release, an agent for improving motor control function, an agent for improving motor learning function and an agent for improving activity motivation, each containing tryptophanylarginine as an active ingredient.

### TECHNICAL FIELD

The present invention relates to techniques for enhancing dopamine release, improving motor control function, improving motor learning function and improving activity motivation (e.g., an agent for enhancing dopamine release, an agent for improving motor control function, an agent for improving motor learning function and an agent for improving activity motivation).

### BACKGROUND ART

Dopamine signaling is known to influence motor control function, motor learning function and activity motivation (Non Patent Literatures 1 and 2).

Patent Literature 1 discloses a composition for improving decreased motivation, depression or depressive mood disorders, containing a decapeptide LSSTQAQQSY, LSSTQAQQSW or LSSTQAQQSF. Patent Literature 1 also discloses production of a decapeptide LSSTQAQQSY by hydrolysis of β-conglycinin, one of soy proteins, with thermolysin. “L” stands for lysine, “S” for serine, “T” for threonine, “Q” for glutamine, “A” for alanine, “Y” for tyrosine, “W” for tryptophan and “F” for phenylalanine.

Patent Literature 2 discloses a composition for improving, maintaining and/or ameliorating motor control function and/or motor learning function, containing a tetrapeptide GTWY. Patent Literature 2 also discloses production of a peptide GTWY through enzymolysis of whey protein. “G” stands for glycine, “T” for threonine, “W” for tryptophan and “Y” for tyrosine.

Patent Literature 3 discloses a composition for mitigation or prevention of a lack of willingness or motivation and/or decrease in vitality, containing a dipeptide LH, DV or MH. Patent Literature 3 also discloses that the effect of these dipeptides is exerted through an action of inhibiting microglial inflammation. Patent Literature 3 also discloses production of a peptide extract containing a dipeptide LH, DV or MH by dissolving non-fat milk powder or defatted soybean protein in water and subjecting it to enzymatic treatment. “L” stands for lysine, “H” for histidine, “D” for aspartic acid, “V” for valine and “M” for methionine.

### CITATION LIST

### Patent Literatures

Patent Literature 1: Japanese Patent Laid-Open No. 2020-040982
Patent Literature 2: Japanese Patent Laid-Open No. 2020-002078
Patent Literature 3: Japanese Patent Laid-Open No. 2016-222646 Non Patent Literatures
Non Patent Literature 1: Clark, B. C., et al., The Aging Brain & the Dorsal Basal Ganglia: Implications for Age-Related Limitations of Mobility., Adv. Geriatr. Med. Res., 2019. 1: e190008.
Non Patent Literature 2: Schultz, W., Predictive reward signal of dopamine neurons., J. Neurophysiol., 1998.80 (1): p. 1-27.

### SUMMARY OF INVENTION

### Technical Problem

An object of the present invention is to provide novel techniques for enhancing dopamine release, improving motor control function, improving motor learning function and improving activity motivation (e.g., a novel agent for enhancing dopamine release, a novel agent for improving motor control function, a novel agent for improving motor learning function and a novel agent for improving activity motivation).

### Solution to Problem

The present invention provides the following agents.
[A1] An agent for enhancing dopamine release, containing tryptophanylarginine as an active ingredient.
[A2] An agent for improving motor control function, containing tryptophanylarginine as an active ingredient.
[A3] The agent for improving motor control function according to [A2], wherein the agent for improving motor control function is used to improve one or more motor control functions selected from motor coordination function, balance function, reaction ability and dexterity.
[A4] An agent for improving motor learning function, containing tryptophanylarginine as an active ingredient.
[A5] An agent for improving activity motivation, containing tryptophanylarginine as an active ingredient.

The present invention also provides the following uses.
[B1] Use of tryptophanylarginine in production of an agent for enhancing dopamine release.
[B2] Use of tryptophanylarginine in production of an agent for improving motor control function.
[B3] The use according to [B2], wherein the agent for improving motor control function is used to improve one or more motor control functions selected from motor coordination function, balance function, reaction ability and dexterity.
[B4] Use of tryptophanylarginine in production of an agent for improving motor learning function.
[B5] Use of tryptophanylarginine in production of an agent for improving activity motivation

The present invention also provides the following methods.
[C1] A method for enhancement of dopamine release, including a step of administering tryptophanylarginine to a subject in need of enhancement of dopamine release.
[C2] A method for improvement of motor control function, including a step of administering tryptophanylarginine to a subject in need of improvement of motor control function.
[C3] The method according to [C2], wherein the improvement of motor control function is improvement of one or more motor control functions selected from motor coordination function, balance function, reaction ability and dexterity.
[C4] A method for improvement of motor learning function, including a step of administering tryptophanylarginine to a subject in need of improvement of motor learning function.
[C5] A method for improvement of activity motivation, including a step of administering tryptophanylarginine to a subject in need of improvement of activity motivation.

The present invention also provides the following uses.
[D1] Use of tryptophanylarginine as an active ingredient in an agent for enhancing dopamine release.
[D2] Use of tryptophanylarginine as an active ingredient in an agent for improving motor control function.
[D3] The use according to [D2], wherein the agent for improving motor control function is used to improve one or more motor control functions selected from motor coordination function, balance function, reaction ability and dexterity.
[D4] Use of tryptophanylarginine as an active ingredient in an agent for improving motor learning function.
[D5] Use of tryptophanylarginine as an active ingredient in an agent for improving activity motivation.

### Effects of Invention

According to the present invention, novel techniques are provided for enhancing dopamine release, improving motor control function, improving motor learning function and improving activity motivation (e.g., a novel agent for enhancing dopamine release, a novel agent for improving motor control function, a novel agent for improving motor learning function and a novel agent for improving activity motivation).

### DESCRIPTION OF EMBODIMENTS

The present invention will be described below.

The present invention provides an agent for enhancing dopamine release (hereinafter may be referred to as “first agent”), an agent for improving motor control function (hereinafter may be referred to as “second agent”), an agent for improving motor learning function (hereinafter may be referred to as “third agent”) and an agent for improving activity motivation (hereinafter may be referred to as “fourth agent”).

### <<Active Ingredient>>

Each of the first to fourth agents contains tryptophanylarginine as an active ingredient. Tryptophanyl arginine is a dipeptide composed of an amino acid sequence represented by WR, wherein “W” stands for tryptophan and “R” stands for arginine. Hereinafter, tryptophanylarginine may be referred to as “dipeptide according to the present invention”.

The dipeptide according to the present invention may be obtained by chemical synthesis or by chemical or enzymatic degradation of a protein. Examples of the protein include a protein derived from soybean (e.g., soybean glycinin). Safety of the dipeptide according to the present invention is supported by abundant food experiences of humanity, as represented by soybeans.

Each of the first to fourth agents may or may not contain a peptide other than the dipeptide according to the present invention, obtained by chemical or enzymatic degradation of a protein (e.g., protein derived from soybean). In one embodiment, each of the first to fourth agents contains no peptide other than the dipeptide according to the present invention, obtained by chemical or enzymatic degradation of a protein (e.g., protein derived from soybean). Examples of the peptide other than the dipeptide according to the present invention, obtained by chemical or enzymatic degradation of a protein (e.g., protein derived from soybean) include a dipeptide WM, a dipeptide LH, DV or MH (Japanese Patent Laid-Open No. 2016-222646), a decapeptide LSSTQAQQSY (Japanese Patent Laid-Open No. 2020-40982) and YPFVV (Japanese Patent Laid-Open No. 2007-91656). “W” stands for tryptophan, “M” for methionine, “L” for lysine, “H” for histidine, “D” for aspartic acid, “V” for valine, “S” for serine, “T” for threonine, “Q” for glutamine, “A” for alanine, “Y” for tyrosine, “P” for proline and “F” for phenylalanine.

Each of the first to fourth agents may or may not contain an active ingredient other than the dipeptide according to the present invention. In one embodiment, each of the first to fourth agents contains the dipeptide according to the present invention as the sole active ingredient (i.e., each of the first to fourth agents contains no active ingredient other than the dipeptide according to the present invention). Examples of a peptide ingredient that is not contained in each of the first to fourth agents include a dipeptide LH, DV or MH (Japanese Patent Laid-Open No. 2016-222646), a tetrapeptide GTWY (Japanese Patent Laid-Open No. 2020-002078), a decapeptide LSSTQAQQSY, LSSTQAQQSW or LSSTQAQQSF (Japanese Patent Laid-Open No. 2020-40982). “L” stands for lysine, “H” for histidine, “D” for aspartic acid, “V” for valine, “M” for methionine, “G” for glycine, “T” for threonine, “W” for tryptophan, “Y” for tyrosine, “S” for serine, “Q” for glutamine, “A” for alanine and “F” for phenylalanine.

The dipeptide according to the present invention may be composed solely of L-amino acids, may be composed solely of D-amino acids, or may be composed of both L- and D-amino acids.

The dipeptide according to the present invention may be in the form of a pharmaceutically acceptable salt or solvate. The expression “each of the first to fourth agents contains tryptophanylarginine as an active ingredient” encompasses, in addition to an embodiment wherein each of the first to fourth agents contains tryptophanylarginine itself, an embodiment wherein each of the first to fourth agents contains tryptophanylarginine in the form of a pharmaceutically acceptable salt and/or solvate thereof. Examples of the pharmaceutically acceptable salt include an acid addition salt, a metal salt, an ammonium salt and an organic amine addition salt. Examples of the pharmaceutically acceptable acid addition salt include inorganic acid salts such as hydrochloride salts, hydrobromide salts, nitrate salts, sulfate salts and phosphate salts, and organic acid salts such as acetate salts, oxalate salts, maleate salts, fumarate salts, citrate salts, benzoate salts and methanesulfonate salts. Examples of the pharmaceutically acceptable metal salt include alkaline metal salts such as sodium salts and potassium salts, alkaline earth metal salts such as magnesium salts and calcium salts, salts of aluminum and salts of zinc. Examples of the pharmaceutically acceptable ammonium salt include salts of ammonium and tetramethylammonium. Examples of the pharmaceutically acceptable organic amine addition salt include addition salts of morpholine and piperidine.

The content of the dipeptide according to the present invention in each of the first to fourth agents can be adjusted as appropriate, taking into consideration the dosage form of each agent, the number of doses of each agent per day, and the dosage of each agent per day. Each of the first to fourth agents may consist solely of the dipeptide according to the present invention. The content of the dipeptide according to the present invention in each of the first to fourth agents can be respectively adjusted to 0.0001% by mass or more to 100% by mass or less, preferably 0.001% by mass or more to 100% by mass or less, based on the mass of each of the first to fourth agents. The content of the dipeptide according to the present invention can be measured by liquid chromatography-tandem mass spectrometry (LC-MS/MS). LC-MS/MS can be performed according to the conditions described in Example.

### <<Subject to Receive Administration>>

The subject to receive administration of the dipeptide according to the present invention or each of the first to fourth agents is preferably a mammal such as human, mouse, rat, hamster, rabbit, dog, cat, hog, cattle, horse or monkey, and more preferably a human.

### <<Applications>>

### <First Application>

The dipeptide according to the present invention exhibits a dopamine release-enhancing effect. In other words, the dipeptide according to the present invention can enhance dopamine release. Therefore, the dipeptide according to the present invention is useful as an active ingredient in an agent for enhancing dopamine release. In addition, the dipeptide according to the present invention can be administered to a subject in need of enhancement of dopamine release, for the purpose of achieving enhancement of dopamine release.

The first agent can enhance dopamine release through the dopamine release-enhancing effect of the dipeptide according to the present invention.

The phrase “enhancement of dopamine release” refers to an increase in the amount of dopamine released (secreted) from a dopamine neuron in the brain. Examples of the dopamine neuron include the cell group A8, the cell group A9, the cell group A10, the cell group A11, the cell group A12, the cell group A13, the cell group A14, the cell group A15, the cell group A16 and the cell group A17. The cell groups A8 to A10 are located in the midbrain, the cell groups A11 to A15 are located in the diencephalon, the cell group A16 is located in the olfactory bulb, and the cell group A17 is located in the retina.

Enhancement of dopamine release can be achieved through promotion of dopamine release from a dopamine neuron in the brain and/or promotion of dopamine production in a dopamine neuron in the brain. Therefore, the dipeptide according to the present invention is useful as an active ingredient in an agent for promoting dopamine release and/or an agent for promoting dopamine production. In addition, the dipeptide according to the present invention can be administered to a subject in need of promotion of dopamine release and/or promotion of dopamine production, for the purpose of achieving promotion of dopamine release and/or promotion of dopamine production.

Enhancement of dopamine release can lead to enhancement of dopamine level in the brain. Therefore, the dipeptide according to the present invention is useful as an active ingredient in an agent for enhancing dopamine level in the brain. In addition, the dipeptide according to the present invention can be administered to a subject in need of enhancement of dopamine level in the brain, for the purpose of achieving enhancement of dopamine level in the brain.

The subject to receive administration of the dipeptide according to the present invention or the first agent is preferably a human in need of enhancement of dopamine release.

In one embodiment, the subject to receive administration is a healthy human. In this embodiment, the dipeptide according to the present invention or the first agent can prevent a reduction in dopamine release or a symptom or disease caused by a reduction in dopamine release in the subject to receive administration, by enhancing dopamine release in the subject to receive administration. The risk of a reduction in dopamine release can increase with age. Therefore, in a preferred embodiment, the subject to receive administration is a healthy human in menopause or old age. The term “menopause” refers to the age range during which a menopausal symptom appears, for example, 45 to 55 years of age. The term “old age” refers to the advanced age following menopause, for example, 56 years of age or older, preferably 60 years of age or older, and more preferably 65 years of age or older.

In another embodiment, the subject to receive administration is a human who exhibits, or is suspected of exhibiting, a reduction in dopamine release. In this embodiment, the dipeptide according to the present invention or the first agent can prevent or ameliorate a reduction in dopamine release or a symptom or disease caused by a reduction in dopamine release in the subject to receive administration, by enhancing dopamine release in the subject to receive administration. The risk of a reduction in dopamine release can increase with age. Therefore, in a preferred embodiment, the subject to receive administration is a human in menopause or old age who exhibits, or is suspected of exhibiting, a reduction in dopamine release. The meanings of the terms “menopause” and “old age” are the same as described above.

The phrase “enhancement of dopamine release” encompasses an enhancement relative to the current state of the subject to receive administration and an enhancement relative to a healthy individual of the same age as the subject to receive administration. The phrase “reduction in dopamine release” encompasses a reduction relative to the current state of the subject to receive administration and a reduction relative to a healthy individual of the same age as the subject to receive administration. The phrase “prevention of a reduction in dopamine release” encompasses maintenance of dopamine release, suppression of a reduction in dopamine release, and delay of a reduction in dopamine release. The phrase “amelioration of a reduction in dopamine release” encompasses restoration of reduced dopamine release and normalization of reduced dopamine release. The phrase “prevention of a symptom or disease” encompasses suppression of onset of a symptom or disease and delay of onset of a symptom or disease. The expression “amelioration of a symptom or disease” encompasses suppression of progression or worsening of a symptom or disease, delay of progression or worsening of a symptom or disease, alleviation of a symptom or disease, and cure of a symptom or disease. Examples of the “symptom or disease caused by a reduction in dopamine release” include tremors in the limbs, postural instability, cognitive impairment and depressive symptoms.

### <Second Application>

The dipeptide according to the present invention exhibits a motor control function-improving effect. In other words, the dipeptide according to the present invention can improve motor control function. Therefore, the dipeptide according to the present invention is useful as an active ingredient in an agent for improving motor control function. In addition, the dipeptide according to the present invention can be administered to a subject in need of improvement of motor control function, for the purpose of achieving improvement of motor control function. The motor control function-improving effect of the dipeptide according to the present invention can be exerted through the dopamine release-enhancing effect of the dipeptide according to the present invention, although the mechanism of action by which the motor control function-improving effect is exerted is not limited thereto.

The second agent can improve motor control function through the motor control function-improving effect of the dipeptide according to the present invention.

The term “motor” refers to actions resulting from voluntary movements (e.g., actions involving voluntary muscles such as those of the limbs and eyes), and encompasses daily activities, sports, playing musical instruments and manual tasks (regardless of whether or not tools are used).

The phrase “motor control function” refers to the function of controlling the movements of skeletal muscles that are consciously activated by the brain (voluntary movements) so that the movements match the targeted movements, i.e., the brain's function of activating skeletal muscles to achieve the targeted actions, and mainly involves feedforward control, which generates motor commands based on the targeted state, and feedback control, which generates motor commands using error signals between the targeted state and the perceived state.

Examples of motor control function include motor coordination function, balance function, reaction ability and dexterity. It is preferable that the second agent is used to improve one or more motor control functions selected from motor coordination function, balance function, reaction ability and dexterity.

The phrase “motor coordination function” refers to the function of coordinating the timing, intensity and the like of multiple muscle activities to enable precise and smooth movements.

The phrase “balance function” refers to the ability to maintain proper body balance based on information such as vision and inner ear sensation.

The phrase “reaction ability” refers to the ability to quickly respond to a given stimulus.

The term “dexterity” refers to the ability to perform dexterous movements using a body, especially the hands, fingers, and the like.

The subject to receive administration of the dipeptide according to the present invention or the second agent is preferably a human in need of improvement of motor control function.

In one embodiment, the subject to receive administration is a healthy human. In this embodiment, the dipeptide according to the present invention or the second agent can prevent a reduction in motor control function in the subject to receive administration, by maintaining or increasing motor control function in the subject to receive administration. The phrase “improvement of motor control function” in the present invention encompasses maintenance of motor control function, an increase in motor control function and prevention of a reduction in motor control function. The risk of a reduction in motor control function can increase with age. Therefore, in a preferred embodiment, the subject to receive administration is a healthy human in menopause or old age. The meanings of the terms “menopause” and “old age” are the same as described above.

In another embodiment, the subject to receive administration is a human who exhibits, or is suspected of exhibiting, a reduction in motor control function. In this embodiment, the dipeptide according to the present invention or the second agent can prevent or ameliorate a reduction in motor control function in the subject to receive administration, by maintaining or increasing motor control function in the subject to receive administration. The phrase “improvement of motor control function” in the present invention encompasses maintenance of motor control function, an increase in motor control function, prevention of a reduction in motor control function and amelioration of reduced motor control function. The risk of a reduction in motor control function can increase with age. Therefore, in a preferred embodiment, the subject to receive administration is a human in menopause or old age who exhibits, or is suspected of exhibiting, a reduction in motor control function. The meanings of the terms “menopause” and “old age” are the same as described above.

The phrase “increase in motor control function” encompasses an increase relative to the current state of the subject to receive administration and an increase relative to a healthy individual of the same age as the subject to receive administration. The phrase “reduction in motor control function” encompasses a reduction relative to the current state of the subject to receive administration and a reduction relative to a healthy individual of the same age as the subject to receive administration. The phrase “prevention of a reduction in motor control function” encompasses maintenance of motor control function, suppression of a reduction in motor control function and delay of a reduction in motor control function. The phrase “amelioration of reduced motor control function” encompasses restoration of reduced motor control function and normalization of reduced motor control function.

### <Third Application>

The dipeptide according to the present invention exhibits a motor learning function-improving effect. In other words, the dipeptide according to the present invention can improve motor learning function. Therefore, the dipeptide according to the present invention is useful as an active ingredient in an agent for improving motor learning function. In addition, the dipeptide according to the present invention can be administered to a subject in need of improvement of motor learning function, for the purpose of achieving improvement of motor learning function. The motor learning function-improving effect of the dipeptide according to the present invention can be exerted through the dopamine release-enhancing effect of the dipeptide according to the present invention, although the mechanism of action by which the motor learning function-improving effect is exerted is not limited thereto.

The third agent can improve motor learning function through the motor learning function-improving effect of the dipeptide according to the present invention.

The phrase “learning function” refers to the function of modifying actions in response to environmental stimuli, i.e., the function of modifying actions through experience, practice, or the like. The “motor learning function” is a subset of the learning function, specifically related to movements.

Examples of motor learning function include motor memory function. The phrase “motor memory function” is a type of implicit memory (unconscious memory that emerges automatically with minimal conscious processing), and refers to the function characterized by sequential learning that progresses with repetition, i.e., the function of storing information related to motor control acquired through experience, practice and the like as internal models of movements. In other words, information related to motor control is accumulated in the brain through motor memory function, and through the motor learning function, the information is built up and movements are updated (becoming more refined and advanced) over time.

The subject to receive administration of the dipeptide according to the present invention or the third agent is preferably a human in need of improvement of motor learning function.

In one embodiment, the subject to receive administration is a healthy human. In this embodiment, the dipeptide according to the present invention or the third agent can prevent a reduction in motor learning function in the subject to receive administration, by maintaining or increasing motor learning function in the subject to receive administration. The phrase “improvement of motor learning function” in the present invention encompasses maintenance of motor learning function, an increase in motor learning function and prevention of a reduction in motor learning function. The risk of a reduction in motor learning function can increase with age. Therefore, in a preferred embodiment, the subject to receive administration is a healthy human in menopause or old age. The meanings of the terms “menopause” and “old age” are the same as described above.

In another embodiment, the subject to receive administration is a human who exhibits, or is suspected of exhibiting, a reduction in motor learning function. In this embodiment, the dipeptide according to the present invention or the third agent can prevent or ameliorate a reduction in motor learning function in the subject to receive administration, by maintaining or increasing motor learning function in the subject to receive administration. The phrase “improvement of motor learning function” in the present invention encompasses maintenance of motor learning function, an increase in motor learning function, prevention of a reduction in motor learning function and amelioration of reduced motor learning function. The risk of a reduction in motor learning function can increase with age. Therefore, in a preferred embodiment, the subject to receive administration is a human in menopause or old age who exhibits, or is suspected of exhibiting, a reduction in motor learning function. The meanings of the terms “menopause” and “old age” are the same as described above.

The phrase “increase in motor learning function” encompasses an increase relative to the current state of the subject to receive administration and an increase relative to a healthy individual of the same age as the subject to receive administration. The phrase “reduction in motor learning function” encompasses a reduction relative to the current state of the subject to receive administration and a reduction relative to a healthy individual of the same age as the subject to receive administration. The phrase “prevention of a reduction in motor learning function” encompasses maintenance of motor learning function, suppression of a reduction in motor learning function and delay of a reduction in motor learning function. The phrase “amelioration of reduced motor learning function” encompasses restoration of reduced motor learning function and normalization of reduced motor learning function.

### <Fourth Application>

The dipeptide according to the present invention exhibits an activity motivation-improving effect. In other words, the dipeptide according to the present invention can improve activity motivation. Therefore, the dipeptide according to the present invention is useful as an active ingredient in an agent for improving activity motivation. In addition, the dipeptide according to the present invention can be administered to a subject in need of improvement of activity motivation, for the purpose of achieving improvement of activity motivation. The activity motivation-improving effect of the dipeptide according to the present invention can be exerted through the dopamine release-enhancing effect of the dipeptide according to the present invention, although the mechanism of action by which the activity motivation-improving effect is exerted is not limited thereto.

The fourth agent can improve activity motivation through the activity motivation-improving effect of the dipeptide according to the present invention.

The term “activity” refers to movements of locomotor system that consume more energy than resting states, and encompasses daily physical activities (actions performed in daily life such as household chores, work, and commuting) and exercise (intentional or planned activities such as sports, aimed at maintaining or increasing physical fitness). The phrase “activity motivation” refers to the psychological drive to voluntarily engage in the “activity” described above.

When activity motivation increases, changes such as more frequent activity, longer activity time, and engagement in activity with greater energy consumption occur, that is, the amount of activity increases. Thus, an increase in activity motivation can be determined by an increase in the amount of activity during the time from waking up to going to bed. The amount of activity can be measured as the total time of voluntary activity or the total energy of voluntary activity during the time from waking up to going to bed.

The subject to receive administration of the dipeptide according to the present invention or the fourth agent is preferably a human in need of improvement of activity motivation.

In one embodiment, the subject to receive administration is a healthy human. In this embodiment, the dipeptide according to the present invention or the fourth agent can prevent a reduction in activity motivation in the subject to receive administration, by maintaining or increasing activity motivation in the subject to receive administration. The phrase “improvement of activity motivation” in the present invention encompasses maintenance of activity motivation, an increase in activity motivation and prevention of a reduction in activity motivation. The risk of a reduction in activity motivation can increase with age. Therefore, in a preferred embodiment, the subject to receive administration is a healthy human in menopause or old age. The meanings of the terms “menopause” and “old age” are the same as described above.

In another embodiment, the subject to receive administration is a human who exhibits, or is suspected of exhibiting, a reduction in activity motivation. In this embodiment, the dipeptide according to the present invention or the fourth agent can prevent or ameliorate a reduction in activity motivation in the subject to receive administration, by maintaining or increasing activity motivation in the subject to receive administration. The phrase “improvement of activity motivation” in the present invention encompasses maintenance of activity motivation, an increase in activity motivation, prevention of a reduction in activity motivation and amelioration of reduced activity motivation. The risk of a reduction in activity motivation can increase with age. Therefore, in a preferred embodiment, the subject to receive administration is a human in menopause or old age who exhibits, or is suspected of exhibiting, a reduction in activity motivation. The meanings of the terms “menopause” and “old age” are the same as described above.

The phrase “increase in activity motivation” encompasses an increase relative to the current state of the subject to receive administration and an increase relative to a healthy individual of the same age as the subject to receive administration. The phrase “reduction in activity motivation” encompasses a reduction relative to the current state of the subject to receive administration and a reduction relative to a healthy individual of the same age as the subject to receive administration. The phrase “prevention of a reduction in activity motivation” encompasses maintenance of activity motivation, suppression of a reduction in activity motivation and delay of a reduction in activity motivation. The phrase “amelioration of a reduction in activity motivation” encompasses restoration of reduced activity motivation and normalization of reduced activity motivation.

According to the fourth agent, a favorable mental and psychological effect on activity can be achieved, which is expected to prevent or improve depressive symptoms or depression that occur during the postmenopausal period, especially in old age. In addition, according to the fourth agent, the amount of activity can be increased, which incidentally suppresses musculoskeletal deterioration and is expected to reduce the risk of requiring long-term care.

The fourth agent exhibits an excellent effect in the first half of action time per day. Therefore, the fourth agent can be used in order to increase the amount of activity in the first half of the action time per day. The phrase “action time per day” refers to a time of day when energy consumption is relatively higher and corresponds to a time when humans are engaged in living activities from waking up to going to bed, i.e., during a daytime. Thus, the fourth agent can be used to increase the amount of activity in the first half of the daytime. According to the fourth agent, the amount of activity in the first half of the action time per day can be increased, which is expected to prevent or improve insomnia that occurs during the postmenopausal period, especially in old age.

### <Dosage and Administration>

The daily dosage of the dipeptide according to the present invention or each of the first to fourth agents can be properly adjusted according to gender, age, weight, symptoms and the like of the subject. The dosage of the dipeptide according to the present invention per adult per day is, for example, 0.01 to 100 mg, preferably 0.1 to 10 mg.

The period of administration, the interval of administration, and the frequency of administration per day of the dipeptide according to the present invention or each of the first to fourth agents can be properly adjusted according to gender, age, weight, symptoms and the like of the subject. The dipeptide according to the present invention or each of the first to fourth agents is administered, for example, daily, every other day or every three days for a period of one week or longer, preferably daily, every other day or every three days for a period of two weeks or longer, more preferably daily, every other day or every three days for a period of four weeks or longer. The frequency of administration per day is not particularly limited, but is typically one, two or three times.

The administration route of the dipeptide according to the present invention or each of the first to fourth agents is not particularly limited, as long as the desired effects (e.g., enhancement of dopamine release, improvement of motor control function, improvement of motor learning function, and improvement of activity motivation) can be achieved. In one embodiment, the administration route is oral administration. In another embodiment, the administration route is parenteral administration. Examples of parenteral administration include intranasal, ophthalmic, otic, transdermal, tracheobronchial, intrarectal, intraurinary, subcutaneous, intramuscular, and intravenous administration. The administration route is preferably oral administration.

### <Dosage Form>

Each of the first to fourth agents may consist solely of the dipeptide according to the present invention, but preferably it is in the form of a composition, more preferably in the form of a food or beverage composition or a pharmaceutical composition.

Examples of the food or beverage composition include health foods, nutritional supplement foods, functional foods, foods with health claims (e.g., foods for specified health uses, foods with nutrient function claims, foods with functional claims), and foods for special dietary uses (e.g., powdered milk for pregnant or lactating women).

Examples of the form of the food or beverage composition include solid, powdered, paste, semi-liquid, gel and liquid forms.

The food or beverage composition is not particularly limited as long as it is a food or beverage that can contain the dipeptide according to the present invention, and examples thereof include instant foods such as instant noodles, retort foods, canned foods, microwavable foods, instant soups/miso soups, and freeze-dried foods; beverages such as soft drinks, fruit juice drinks, vegetable drinks, soy milk drinks, coffee drinks, tea drinks, powdered drinks, concentrated drinks, and alcoholic drinks; flour products such as bread, pasta, noodles, cake mixes, and bread crumbs; confections such as candies, caramels, chewing gum, chocolate, cookies, biscuits, bars, cakes, pies, snacks, crackers, Japanese sweets, mousse, and dessert sweets; condiments such as sauces, tomato-processed seasonings, flavoring seasonings, cooking mixes, liquid condiments, dressings, soups, and curry and stew bases; fats and oils such as processed fats and oils, butter, margarine, and mayonnaise; dairy products such as milk drinks, dairy drinks, fermented milk, cheese, yogurt, drinks containing lactic acid-producing bacteria, dairy drinks, cheese, ice cream, cream, modified milk powder, liquid milk, and solid milk; processed agricultural products such as canned agricultural foods, jam/marmalade, and cereal; frozen foods, liquid diet, and supplements. The food or beverage composition can be produced according to a conventional method for production, except that the dipeptide according to the present invention is contained.

Ingredients other than the dipeptide according to the present invention contained in the food or beverage composition are not particularly limited. Examples of ingredients other than the dipeptide according to the present invention include water, proteins, carbohydrates, lipids, vitamins, minerals, organic acids, organic bases, fruit juices and flavors. Examples of proteins include animal and vegetable proteins such as whole milk powder, skimmed milk powder, partially skimmed milk powder, casein, whey powder, whey protein, whey protein concentrate, whey protein isolate, α-casein, β-casein, κ-casein, β-lactoglobulin, α-lactalbumin, lactoferrin, soy protein, hen egg protein, and meat protein; hydrolysates of such proteins; and various milk-derived components such as butter, whey minerals, cream, whey, non-protein nitrogen, sialic acid, phospholipid, and lactose. Examples of carbohydrates include commonly used sugars, modified starch (such as dextrin, soluble starch, British starch, oxidized starch, starch ester, and starch ether), and dietary fiber. Examples of lipids include animal fats and oils such as lard, fish oil, and their fractionated oils, hydrogenated oils, and ester-exchanged oils; and vegetable fats and oils such as palm oil, safflower oil, corn oil, rapeseed oil, coconut oil, and their fractionated oils, hydrogenated oils, and ester-exchanged oils. Examples of vitamins include vitamin A, carotenes, vitamin B complex, vitamin C, vitamin D complex, vitamin E, vitamin K complex, vitamin P, vitamin Q, niacin, nicotinic acid, pantothenic acid, biotin, inositol, choline, and folic acid, and examples of minerals include calcium, potassium, magnesium, sodium, copper, iron, manganese, zinc, selenium, and whey minerals. Examples of organic acids include malic acid, citric acid, lactic acid, and tartaric acid. One of such ingredients may be used alone, or two or more of such ingredients may be used in combination.

The pharmaceutical composition contains the dipeptide according to the present invention and one or more pharmaceutically acceptable carriers. The pharmaceutical composition is preferably a pharmaceutical composition administered orally (oral formulation). Examples of the pharmaceutical composition include granules, powders, tablets (including sugar-coated tablets), pills, capsules, syrups, emulsions and suspensions. These formulations can be produced using a pharmaceutically acceptable carrier according to a conventional method. Examples of the pharmaceutically acceptable carrier include excipients, binders, diluents, additives, flavoring agents, buffers, thickeners, colorants, stabilizers, emulsifiers, dispersants, suspending agents and preservatives. A single pharmaceutically acceptable carrier may be used alone, or two or more pharmaceutically acceptable carriers may be used in combination.

Each of the first to fourth agents may be packaged to facilitate administration of a single dose. A single dose may be in a single package or in multiple packages. When provided in a packaged form, each of the agents is preferably provided in a form that facilitates continuous consumption, such as a set containing an amount to be consumed over a specific period of time (for example, an amount to be consumed in several days). The package form is not particularly limited as long as a specific amount is provided, and examples include wrapping paper, sacks, soft bags, paper containers, cans, bottles, and capsules.

The application, dosage, administration method, and the like of each of the first to fourth agents may be displayed on the packaging, container, or package insert (such as a product description, a pamphlet, a promotion item, and a brand site). Here, the term “display” includes all sorts of display intended to inform consumers of the above application. Such display may be any display that enables the above application, dosage, administration method, and the like to be recalled or inferred, and may include all sorts of display irrespectively of the purpose of display, contents of display, displayed object, medium, and the like.

### EXAMPLES

The present invention will be described in detail below based on Examples, but the invention is not limited to the Examples.

### Examples and Comparative Examples

### <Materials>

PC12 cells were obtained from RIKEN (Saitama, Japan) and used within 20 passages.

DMEM (Dulbecco's Modified Eagle's Medium), penicillin and streptomycin were purchased from Gibco (Carlsbad, CA), FBS (Fetal Bovine Serum) from Biological Industries (Kibbutz Beit-Haemek, Israel) and HS (Horse Serum) from Gibco (Carlsbad, CA).

Dipeptides (tryptophanylarginine (WR), tryptophanylphenylalanine (WF), tryptophanylhistidine (WH) and tryptophanylmethionine (WM)) were purchased from GenScript (Piscataway, NJ). All the dipeptides are synthetic dipeptides. “W” stands for tryptophan, “R” for arginine, “F” for phenylalanine, “H” for histidine, and “M” for methionine.

### <PC12 Cell Culture>

PC12 cells were seeded in a 96-well plate at a cell count of 2×10⁴ cells/well using DMEM containing 10% by volume of HS (Horse Serum), 10% by volume of FBS (Fetal Bovine Serum), 100 units/mL of penicillin and 100 μg/mL of streptomycin. The cells were cultured at 5% CO₂ by volume and 37° C. The cells were cultured for 3 to 5 days to ensure that they were confluent, and then used for experiments.

### <Dipeptide Addition to PC12 Cells>

The culture medium was removed from PC12 cells, which were washed twice with PBS (Phosphate Buffered Saline). The wells of the 96-well plate were divided into 5 groups: non-stimulated group (5 wells), WF administration group (3 wells), WH administration group (3 wells), WM administration group (3 wells) and WR administration group (3 wells). The WR administration group corresponds to Inventive Example, and the non-stimulated group, WF administration group, WH administration group and WM administration group correspond to Comparative Examples.

DMSO (Dimethyl Sulfoxide) was added to the same medium as the medium used to culture PC12 cells, thereby preparing a medium containing 1% by mass of DMSO. Dipeptide WF was added to the same medium as the medium used to culture PC12 cells, thereby preparing a medium containing 1 mM dipeptide WF. In the same way, a medium containing 1 mM dipeptide WH, a medium containing 1 mM dipeptide WM and a medium containing 1 mM dipeptide WR were prepared. The medium containing 1% by volume of DMSO (Dimethyl Sulfoxide) was added to the non-stimulated group, the medium containing 1 mM dipeptide WF was added to the WF administration group, the medium containing 1 mM dipeptide WH was added to the WH administration group, the medium containing 1 mM dipeptide WM was added to the WM administration group, and the medium containing 1 mM dipeptide WR was added to the WR administration group. These groups were incubated at 37° C. for 6 hours. After the incubation, supernatant of each well was collected.

### <Sample Preparation>

Each supernatant was transferred onto ice. To each supernatant, 25 μL of 2M perchloric acid was added for protein removal, and after 10 minutes, 50 μL of 1M sodium acetate was added for neutralization. After 15 minutes, centrifugation was performed at 4° C. and 20000 G for 20 minutes. 100 μL of each supernatant was collected, and after added with 10 μL of 2 mM 3,4-dihydroxybenzylamine (DHBA) as an internal standard, it was passed through a chromatodisk to obtain a sample. The obtained sample was used for liquid chromatography-tandem mass spectrometry (LC-MS/MS).

### <LC-MS/MS>

LC-MS/MS was performed using a Triple TOF 6600+ system manufactured by SCIEX under the following measurement conditions. A calibration curve was prepared using pure products, and after correcting the peak area of dopamine with the peak area of the internal standard (DHBA), the dopamine concentration (nM) in each sample was calculated from the calibration curve.
Equipment: Triple TOF 6600+ System (SCIEX, Massachusetts, USA)
Column: Inertsil (registered trademark) ODS-4 (GL Sciences, Tokyo, Japan)
Eluent A: 0.1% aqueous solution of formic acid
Eluent B: 0.1% methanol solution of formic acid
Gradient conditions: As listed in Table 1

TABLE 1

| Time | Eluent A | Eluent B |
|---|---|---|
| (minutes) | (% by volume) | (% by volume) |
| 0~10 minutes | 98% | 2% |
| 11~12 minutes | 2% | 98% |
| 13~14 minutes | 98% | 2% |

Dopamine m/z values: Q1 154.1, Q3 90.9
Internal standard (DHBA) m/z values: Q1 140.1, Q3 123.0

### MS Conditions

declustering potential: 37 V
collision energy: 8 V
curtain gas: 20 psi
gas 1:50 psi
gas 2:50 psi
Ion spray voltage: 5500V

“Q1” means a precursor ion, and “Q3” means a product ion generated by collision of a precursor ion with gas. That is, for dopamine, a precursor ion with m/z of 154.1 was selected and collided with gas, and a product ion with m/z of 90.9 was detected. For the internal standard (DHBA), a precursor ion with m/z of 140.1 was selected and collided with gas, and a product ion with m/z of 123.0 was detected.

### <Statistical Processing>

Data are presented as mean±standard deviation. For statistical processing, Student's t-test was used for comparisons between the non-stimulated group and each administration group. The significance level was set at 5%.

### <Results>

The measurement result of dopamine concentration in each sample is shown in Table 2. As shown in Table 2, the WR administration group exhibited a significant increase in dopamine concentration compared to the non-stimulated group (p<0.05). In contrast, the WF administration group, WH administration group and WM administration group did not exhibit such a significant increase in dopamine concentration.

TABLE 2

| | | Dopamine | | Percentage |
|---|---|---|---|---|
| | | concentration | Mean ± | relative to |
| | Well | after stimu- | Standard | non-stimulated |
| | No. | lation (nM) | deviation | group |
| Non- | 1 | 26.93 | 23.50 ± 6.69 | 100 |
| stimulated | 2 | 17.59 | | |
| group | 3 | 17.09 | | |
| | 4 | 22.86 | | |
| | 5 | 33.02 | | |
| WF | 1 | 18.97 | 19.56 ± 1.35 | 83.23 |
| administration | 2 | 18.60 | | |
| group | 3 | 21.10 | | |
| WH | 1 | 26.89 | 22.15 ± 4.13 | 94.26 |
| administration | 2 | 19.36 | | |
| group | 3 | 20.20 | | |
| WM | 1 | 20.58 | 24.57 ± 7.88 | 104.55 |
| administration | 2 | 19.48 | | |
| group | 3 | 33.64 | | |
| WR | 1 | 38.47 | 36.24 ± 2.10* | 154.23 |
| administration | 2 | 34.30 | | |
| group | 3 | 35.95 | | |

(*p < 0.05, Student's t-test)

1.-5. (canceled)

6. A method for enhancement of dopamine release, comprising administering tryptophanylarginine to a subject in need thereof.

7. A method for improvement of motor control function, comprising administering tryptophanylarginine to a subject in need thereof.

8. The method according to claim 7, wherein the improvement of motor control function is improvement of one or more motor control functions selected from motor coordination function, balance function, reaction ability and dexterity.

9. A method for improvement of motor learning function, comprising administering tryptophanylarginine to a subject in need thereof.

10. A method for improvement of activity motivation, comprising administering tryptophanylarginine to a subject in need thereof.
